(19) United States
(12) Patent Application Publication
(10) Pub. No.: US 2025/0090360 A1
(43) Pub. Date: Mar. 20, 2025

(54) WEARING-TYPE CORRECTING APPARATUS FOR HUMAN BODY AND CORRECTION MONITORING METHOD USING SAME

(71) Applicant: Value&Trust, Daegu (KR)

(72) Inventor: Kyung Suk ROE, Seoul (KR)

(73) Assignee: Value&Trust, Daegu (KR)

(21) Appl. No.: 18/969,183

(22) Filed: Dec. 4, 2024

Related U.S. Application Data
(63) Continuation of application No. PCT/KR2022/008741, filed on Jun. 21, 2022.

(30) Foreign Application Priority Data
Jun. 20, 2022 (KR) 10-2022-0074710

(51) Int. Cl. A61F 5/02; A61F 5/01

(52) U.S. Cl. CPC A61F 5/026; A61F 5/0104; A61F 2005/0188

(57) ABSTRACT

Provided is a wearing-type correction apparatus for the human body, wherein the apparatus can accurately detect compression intensity at a position close to a curved portion of the body to be corrected, and show data including the compression intensity to a user via a correction management service application installed in a user terminal. To this end, the wearing-type correcting apparatus for the human body according to the present invention comprises: clothing worn on the upper body of a patient; a curve compressor that is disposed on the clothing and compresses the curve portion to be corrected; an intensity adjuster that is disposed on the clothing and connected to the curve compressor by a wire, and that adjusts the compression intensity of the curve compressor by adjusting the amount of winding and unwinding of the wire; and a pressure sensor installed in at least one of the curve compressor or the intensity adjuster to sense the compression intensity.

### TECHNICAL FIELD

The present disclosure relates to a wearing type correction apparatus for a human body and a method of monitoring corrections using the same, and more particularly, to a wearing type correction apparatus for a human body that is worn on an upper body of a patient and that corrects a curved part of a human body, that is, a target to be corrected, and a hump part by compressing the curved part and the hump part and a method of monitoring corrections using the same.

### BACKGROUND ART

In general, a spine refers to a bone that maintains a main frame including neck, back, waist, hip, and tailbone parts in a person. Specifically, the spine is divided into seven neck bone (cervical vertebra), twelve breastbones (thoracic vertebrae), five lumbar vertebraes (lumbar vertebrae), five bones in the lower back (sacral vertebrae), and four tailbones (coccygeal vertebra).

The spine includes a spinal cord as a bundle of nerves that comes from the brain. The spinal cord performs a role to connect the brain, that is, a central nervous system, and distal organs, that is, peripheral nervous systems. As described above, the spine is a very important portion of a human body, and a person has a lot of difficulties in life if he or she suffers from a spine disease.

Representative spine diseases include cervical vertebral disc herniation, scoliosis, failed back surgery, intervertebral cartilage disc herniation, etc.

In particular, the scoliosis is one of representative spine deformities, and causes a “curve” meaning “the state of being curved sideways” or “the state of being bent sideways” in the spine of a person.

Such scoliosis occurs a lot in the growing period of teenagers to the extent that about half the total of scoliosis patients is the teenagers.

Other measures need to be taken depending on a degree of scoliosis, but there are frequent cases of aggravation because a disease symbol is not recognized or only simple observation measures are taken.

Accordingly, the purposes of treatment of scoliosis are to prevent a mild curve from proceeding, to correct a curve having severity or more, and to balance a human body and improve beauty by maintaining the corrections.

If scoliosis is discovered in early stages, a progress into a severe curve can be prevented even by a conservative method, such as the wearing of a proper assister.

However, a conventional assist device has a problem in that it does not effectively handle scoliosis depending on a degree of the deformity of a scoliosis patient. In particular, there is a problem in that spine corrections are not effectively performed in a process of the youth growing because the youth continue to grow.

Moreover, there is a problem in that a patient who uses an inconvenient assist device gives up the use of the assist device easily because the patient is not sufficiently motivated according to the use of assist devices.

A “spine assister” is disclosed in Korean Patent No. 10-1070973 (Oct. 6, 2011) (hereinafter referred to as a “conventional technology”). The conventional technology relates to a spine assister that is worn by a patient for spine corrections and recovery with respect to spine diseases, such as a spine curve.

The conventional technology includes a back part the length and angle of which area adjustable and the inner/outer skins of which are formed of functional fiber, and a waistband part that is detached from/attached to the back part and that has a pull cord fixture constructed therein so that the waistband part can be fastened by a simple manipulation.

However, the conventional technology has a structure that makes it very difficult for a patient to perform adjustment using the pull cord adjuster for himself or herself, and has a problem in that a person must ask for another person's help when using the spine assister.

Furthermore, scoliosis also causes “hump”, that is, a part that protrudes to the back of a human body, in addition to the “curve”, depending on a patient. The locations of the curve and the hump are different from each other depending on a patient. However, the conventional technology has a problem in that locations at which the curve and the hump are compressed cannot be adjusted.

Furthermore, there are problems in that a user does not know how much the curve or the hump should be compressed with some compression intensity and does not know whether the user observes a compression time of the curve or the hump.

### DISCLOSURE

### Technical Problem

An object of the present disclosure is to provide a wearing type correction apparatus for a human body which can accurately detect compression intensity at a location close to a curved part of a human body, that is, a target to be corrected, and show data including the compression intensity to a user through a correction management service app installed in a user terminal.

Another object of the present disclosure is to provide a wearing type correction apparatus for a human body which can motivate a patient to continuously wear the correction apparatus through the correction management service app, can allow a guardian and a doctor to check whether a patient observes the wearing of the correction apparatus according to the prescription of a doctor, and can allow a doctor to view weekly and monthly reports for a patient's wearing of the correction apparatus, in particular.

Another object of the present disclosure is to provide a wearing type correction apparatus for a human body which enables a compression location to be adjusted based on the location of a curved part and allows a patient to easily adjust the intensity of compression for the curved part for himself or herself.

Furthermore, an additional object of the present disclosure is to provide a method of monitoring corrections which can monitor the wearing state of a patient by using compression intensity (a compression rate) detected by a compression sensor, a wearing time calculated based on the compression intensity, and compliance calculated based on the compression intensity and the wearing time.

Technical objects of the present disclosure are not limited to the aforementioned objects, and the other objects not described above may be evidently understood from the following description by those skilled in the art.

### Technical Solution

In order to achieve the objects, a wearing type correction apparatus for a human body according to an embodiment of the present disclosure consists of clothing, a curve compressor, an intensity adjuster, and a pressure sensor. The clothing may be worn on the upper body of a patient. The curve compressor may be disposed in the clothing. The curve compressor may compress a curved part of a human body, that is, a target to be corrected. The intensity adjuster may be disposed in the clothing. The intensity adjuster may be connected to the curve compressor through a wire. The intensity adjuster may adjust the compression intensity of the curve compressor by adjusting the amount of winding and unwinding of the wire. The pressure sensor may be installed in at least one of the curve compressor and the intensity adjuster, and may detect the compression intensity.

A short-range wireless communication unit may be further installed in at least one of the curve compressor and the intensity adjuster. The short-range wireless communication unit may communicate with a user terminal in which a correction management service app for informing a user of data including the compression intensity detected by the pressure sensor has been installed.

A guide rail may be further disposed in the clothing. The guide rail may provide guidance to the up and down sliding movements of at least one of the curve compressor and the intensity adjuster.

At least one of the curve compressor and the intensity adjuster may consist of a body, a support plate, and a rail moving block. The body may form an appearance shape of at least one of the curve compressor and the intensity adjuster. The support plate may be disposed between the body and the guide rail. The support plate may be combined with the body. The rail moving block may be combined with the support plate. The rail moving block may be slidably combined with the guide rail.

A dial adjuster may be further constructed in the intensity adjuster. The dial adjuster may be rotatably combined with the body, and may adjust the amount of winding and unwinding of the wire.

A first coupling hole and a second coupling hole may be formed in the support plate. The second coupling hole may extend from the first coupling hole in the sliding direction of the rail moving block. The second coupling hole may be formed to have a smaller width than the first coupling hole in a direction orthogonal to the sliding direction of the rail moving block. The rail moving block may be fit into the first coupling hole and may be then slid and move to the second coupling hole and combined with the support plate.

The rail moving block may consist of a locking part, a support part, and a coupling part. The locking part may be formed to have a width equal to or smaller than the width of the first coupling hole in the direction orthogonal to the sliding direction of the rail moving block. The locking part may be formed to have a width greater than the width of the second coupling hole in the direction orthogonal to the sliding direction of the rail moving block. One surface of the locking part may come into contact with the outer surface of the support plate. The support part may be formed to have a width greater than the width of each of the first coupling hole and the second coupling hole in the direction orthogonal to the sliding direction of the rail moving block. One surface of the support part may face the one surface of the locking part. The one surface of the support part may come into contact with an inner surface of the support plate. The coupling part may be formed to protrude on the other surface of the support part. A guide groove that is slidably combined with the guide rail may be formed in the coupling part.

The guide rail may consist of an attachment plate and a guide protrusion. The attachment plate may be combined with the clothing. The guide protrusion may be formed on one side of the attachment plate in the direction orthogonal to the sliding direction of the rail moving block. The guide protrusion may be inserted into the guide groove and rotatably disposed therein.

The guide groove and the guide protrusion may each be formed to have a circular cross section.

An opening through which one side of the guide groove is opened may be formed in the coupling part. The opening width of the opening may be formed to be smaller than the diameter of the guide groove.

A holder fixing pin may be further constructed in the intensity adjuster. The holder fixing pin may combine the body and the support plate. A locking protrusion may be formed in the outer circumferential surface of the holder fixing pin. A through hole may be formed in the body. A boss part may be formed in the support plate. The boss part may be inserted into the through hole. A holder fixing pin may be inserted into the boss part. A locking groove that is locked in the locking protrusion may be formed within the boss part.

A pelvis band may be further constructed in the wearing type correction apparatus for a human body according to an embodiment of the present disclosure. The pelvis band may be disposed in the clothing. The pelvis band may hold the pelvis of the patient when the curve compressor compresses the curved part.

A back reinforcement plate and a hump pad may be further constructed in the wearing type correction apparatus for a human body according to an embodiment of the present disclosure. The back reinforcement plate may be disposed on an inner surface of the back part of the clothing. The hump pad may be attached to an arbitrary location related to a location of the back reinforcement plate at which the curve compressor compresses the curved part, and may compress the hump part of the human body.

A flank band may be further constructed in the wearing type correction apparatus for a human body according to an embodiment of the present disclosure. The flank band may be disposed in the clothing. The flank band may hold the flank of the patient when the curve compressor compresses the curved part.

A flank pad may be further constructed in the wearing type correction apparatus for a human body according to an embodiment of the present disclosure. The flank pad may be attached to an arbitrary location related to a location of the flank band at which the curve compressor compresses the curved part, and may compress the body.

The hump pad and the flank pad may each consist of a base and a cushion protrusion. The base may be formed in a sheet form. The base may be attached to the back reinforcement plate or the flank band. The cushion protrusion may be formed to protrude on one surface of the base. The cushion protrusion may have cushion power that compresses the body. A wire of a metal material may be disposed within the edge of the base.

A point band may be further constructed in the wearing type correction apparatus for a human body according to an embodiment of the present disclosure. The point band may be attached to an arbitrary location of the clothing. A plurality of metal points that are spaced apart from each other up and down may be disposed in the point band.

A zipper may be installed in the middle of the front part of the clothing from the top to the bottom thereof.

Meanwhile, a method of monitoring corrections using a wearing type correction apparatus for a human body according to an embodiment of the present disclosure may include a clothing wearing detection step of detecting, by a pressure sensor installed in at least one of a curve compressor and an intensity adjuster disposed in clothing, first reference pressure in a state in which a patient has worn the clothing, a band wearing detection step of detecting, by the pressure sensor, second reference pressure in a state in which the patient has worn at least one band disposed in the clothing, a compression intensity detection step of measuring, by the pressure sensor, compression intensity at predetermined time intervals after the first reference pressure and the second reference pressure are detected, and a display step of visually displaying the compression intensity through a user terminal.

Furthermore, the method of monitoring corrections using a wearing type correction apparatus for a human body according to an embodiment of the present disclosure may further include a pressurization state analysis step of analyzing, by at least one of the user terminal and a server that communicates with the user terminal, a pressurization state of the patient based on the compression intensity. The pressurization state may include at least three states based on the first reference pressure and the second reference pressure.

Furthermore, the pressurization state may include an excessive compression state in which the compression intensity is equal to or greater than maximum pressure at which the patient does not suffer from a pain, a normal compression state in which the compression intensity is equal to or greater than average pressure that is an average value of the first reference pressure and the second reference pressure and is less than the maximum pressure, a band non-wearing state in which the compression intensity is equal to or greater than minimum pressure obtained by subtracting, from the first reference pressure, a deviation between the first reference pressure and the average pressure and is less than the average pressure, and an apparatus non-wearing state in which the compression intensity is less than the minimum pressure.

Furthermore, the method of monitoring corrections using a wearing type correction apparatus for a human body according to an embodiment of the present disclosure may further include a prescription pressure input step of receiving, from the user terminal, prescription pressure separately from the first reference pressure and the second reference pressure. The pressurization state may include an excessive compression state in which the compression intensity is equal to or greater than maximum pressure at which the patient does not suffer from a pain, a normal compression state in which the compression intensity is equal to or greater than the prescription pressure and is less than the maximum pressure, a band non-wearing state in which the compression intensity is equal to or greater than minimum pressure obtained by subtracting, from the first reference pressure, a deviation between the first reference pressure and average pressure that is an average value of the first reference pressure and the second reference pressure and is less than the prescription pressure, and an apparatus non-wearing state in which the compression intensity is less than the minimum pressure.

Furthermore, in the display step, a wearing time calculated by using the compression intensity may also be displayed. The wearing time may be calculated by adding times for which the pressurization state is the normal compression state and the band non-wearing state.

Furthermore, the display step may include additionally outputting a guide message for improving the pressurization state of the patient, based on compliance calculated based on the compression intensity and the wearing time.

Furthermore, the method of monitoring corrections using a wearing type correction apparatus for a human body according to an embodiment of the present disclosure may further include a releasing step of adjusting the compression intensity of the curve compressor by adjusting an amount of winding and unwinding of a wire that connects the intensity adjuster and the curve compressor through control of at least one of the curve compressor and the intensity adjuster when the pressurization state is analyzed as the excessive compression state.

The details of other embodiments are included in the detailed description and drawings.

### Advantageous Effects

The wearing type correction apparatus for a human body according to an embodiment of the present disclosure has effects in that the pressure sensor can accurately detect compression intensity because the pressure sensor is installed in at least one of the curve compressor and the intensity adjuster, that is, a location close to a curved part of a human body, that is, a target to be corrected, and data including the compression intensity detected by the pressure sensor can be showed to a user through the correction management service app installed in the user terminal.

Furthermore, the wearing type correction apparatus for a human body according to an embodiment of the present disclosure has an effect in that a curved part of a patient can be corrected by adjusting up and down locations of the curve compressor based on the location of the curved part because the up and down locations of the curve compressor that compresses the curved part of a human body, that is, a target to be corrected, can be adjusted.

Furthermore, the wearing type correction apparatus for a human body according to an embodiment of the present disclosure has an effect in that a patient can easily adjust the compression intensity of the curve compressor for himself or herself by manipulating the intensity adjuster because up and down locations of the intensity adjuster that adjusts the compression intensity of the curve compressor can be adjusted.

Furthermore, the wearing type correction apparatus for a human body according to an embodiment of the present disclosure has an effect in that efficiency of the curve compressor that compresses the curved part can be improved because the pelvis band holds the pelvis of a patient when the curve compressor compresses the curved part.

Furthermore, the wearing type correction apparatus for a human body according to an embodiment of the present disclosure has an effect in that efficiency of the curve compressor that compresses the curved part can be improved because the hump pad is attached to an arbitrary location related to a location at which the curve compressor compresses the curved part and the hump part of the human body that protrudes to the arbitrary location is compressed by the hump pad when the curve compressor compresses the curved part.

Furthermore, the wearing type correction apparatus for a human body according to an embodiment of the present disclosure has an effect in that efficiency of the curve compressor that compresses the curved part can be improved because the flank band holds the flank of a patient when the curve compressor compresses the curved part.

Furthermore, the wearing type correction apparatus for a human body according to an embodiment of the present disclosure has an effect in that efficiency of the curve compressor that compresses the curved part can be improved because the flank pad is attached to an arbitrary location related to a location at which the curve compressor compresses the curved part and the flank pad compresses the arbitrary location when the curve compressor compresses the curved part.

Furthermore, the wearing type correction apparatus for a human body according to an embodiment of the present disclosure has an effect in that a doctor can easily treat the locations and states of the curved part and the hump part of a patient by monitoring the wire and the metal point in an X-ray photo because the wire of a metal material is disposed in the hump pad and the flank pad, the plurality of metal points is disposed in the point band, and thus the wire of a metal material and the metal points are displayed in the X-ray photo that is photographed with the human body correction apparatus worn.

Furthermore, in the method of monitoring corrections using a wearing type correction apparatus for a human body according to an embodiment of the present disclosure, whether a patient wears the wearing type correction apparatus for a human body normally can be easily identified by analyzing compression intensity measured by the pressure sensor. A doctor can establish a corresponding suitable treatment plan.

Furthermore, in the method of monitoring corrections using a wearing type correction apparatus for a human body according to an embodiment of the present disclosure, personalization and optimized monitoring can be performed on a patient because prescription pressure is input and a pressurization state is divided and analyzed based on the prescription pressure.

Furthermore, in the method of monitoring corrections using a wearing type correction apparatus for a human body according to an embodiment of the present disclosure, a patient can be induced to correctly wear the apparatus because a guide message capable of improving the pressurization state of the patient is output based on compliance that is calculated based on compression intensity and a wearing time.

Furthermore, in the method of monitoring corrections using a wearing type correction apparatus for a human body according to an embodiment of the present disclosure, when it is determined that excessive compression intensity is applied to the human body of a patient, inconvenience of the patient can be minimized by adjusting the amount of winding and unwinding of the wire through control of at least one of the curve compressor 100a and the intensity adjuster 100.

Effects of the present disclosure are not limited to the aforementioned effects, and the other effects not described above may be evidently understood by those skilled in the art from the claims.

### DESCRIPTION OF DRAWINGS

FIG. 1 is a front perspective view illustrating a wearing type correction apparatus for a human body according to an embodiment of the present disclosure.

FIG. 2 is a rear perspective view illustrating a wearing type correction apparatus for a human body according to an embodiment of the present disclosure.

FIG. 3 is an exploded perspective view illustrating an installation structure of an intensity adjuster illustrated in FIGS. 1 and 2.

FIG. 4 is an exploded perspective view except the body in FIG. 3.

FIG. 5 is a cross-sectional view taken along line A-A of FIG. 3.

FIGS. 6 and 7 are coupling sequence diagrams of the body and the support plate illustrated in FIG. 3.

FIG. 8 is a diagram illustrating an inner surface of clothing illustrated in FIG. 1.

FIG. 9 is a perspective view illustrating a hump pad illustrated in FIG. 8.

FIG. 10 is a perspective view illustrating a flank pad illustrated in FIG. 8.

FIG. 11 is a diagram illustrating the state in which a point band has been attached to FIG. 1.

FIG. 12 is a control block diagram of the wearing type correction apparatus for a human body according to an embodiment of the present disclosure.

FIG. 13 is a diagram illustrating the state in which a correction management service app installed in a user terminal illustrated in FIG. 12 is displayed on a screen of the user terminal.

FIG. 14 is a communication state diagram of the wearing type correction apparatus for a human body according to an embodiment of the present disclosure.

FIG. 15 is an exemplary flowchart of a method of monitoring corrections using a wearing type correction apparatus for a human body according to an embodiment of the present disclosure.

FIG. 16 is a diagram for describing a process of detecting first reference pressure and second reference pressure in the method of monitoring corrections according to FIG. 15.

FIG. 17 is a diagram for describing average pressure and minimum pressure in the method of monitoring corrections according to FIG. 15.

FIG. 18 is a diagram illustrating a pressurization state that is analyzed based on measured compression intensity.

FIG. 19 is a diagram illustrating a pressurization state that is analyzed based on compression intensity measured through prescription pressure.

FIG. 20 is a diagram illustrating a daily compression rate graph that is displayed on the user terminal.

FIG. 21 is a diagram illustrating a daily wearing time graph that is displayed on the user terminal.

<Description of Reference Numerals>

| | |
|---|---|
| 10: clothing | 20: zipper |
| 100: intensity adjuster | 100a: curve compressor |
| 101: pressure sensor | 102: short-range wireless |
| | communication unit |
| 105: wire | 110: body |
| 113: through hole | 117: holder fixing pin |
| 117a: locking protrusion | 120: dial adjuster |
| 140: support plate | 143: boss part |
| 143a: locking groove | 145: rail moving block |
| 145a: locking part | 145b: support part |
| 145c: coupling part | 145e: guide groove |
| 145f: opening | 147a: first coupling hole |
| 147b: second coupling hole | 150: guide rail |
| 151: attachment plate | 152: guide protrusion |
| 160: pelvis band | 170: back reinforcement plate |
| 180: hump pad | 181, 201: base |
| 182, 202: cushion protrusion | 183, 203: wire |
| 190: flank band | 200: flank pad |
| 210: point band | 215: metal point |
| 300, 400, 500: user terminal | |
| S100: prescription pressure | S110: clothing wearing |
| input step | detection step |
| S120: band wearing detection | S130: compression intensity |
| step | detection step |
| S140: pressurization state | S150: display step |
| analysis step | |
| S160: releasing step | |
| P1: first reference pressure | P2: second reference pressure |
| Pavg: average pressure | Ps: prescription pressure |
| PMAX: maximum pressure | Pmin: minimum pressure |

### BEST MODE

Hereinafter, a wearing type correction apparatus for a human body according to an embodiment of the present disclosure is described with reference to the drawings.

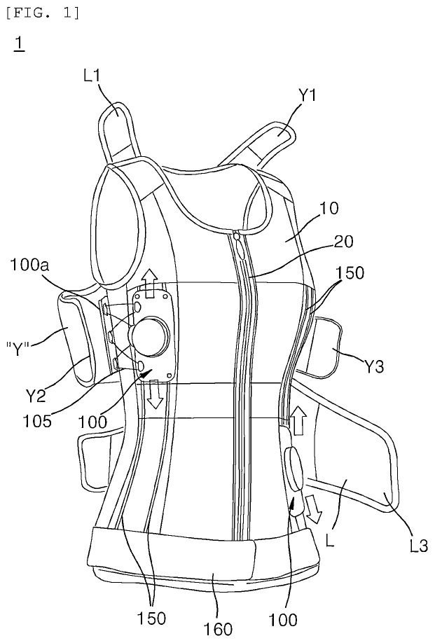

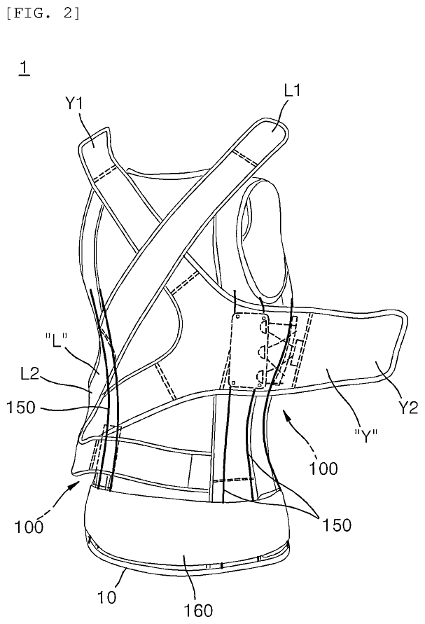

FIG. 1 is a front perspective view illustrating a wearing type correction apparatus for a human body according to an embodiment of the present disclosure. FIG. 2 is a rear perspective view illustrating a wearing type correction apparatus for a human body according to an embodiment of the present disclosure.

Referring to FIGS. 1 and 2, a wearing type correction apparatus 1 for a human body according to an embodiment of the present disclosure may include clothing 10, a curve compressor 100a, and an intensity adjuster 100.

The clothing 10 may be constituted with the top that is worn on an upper body of a patient. The clothing 10 may be formed in the form of a vest (sleeveless shirt) that is worn on the upper body of the patient. The clothing 10 does not need to be essentially formed in a vest form, and may be constituted with a top having various forms, such as short sleeves or long sleeves. The clothing 10 may be formed of a textile material. The clothing 10 may be formed of a synthetic fiber material.

The wearing type correction apparatus 1 for a human body according to an embodiment of the present disclosure includes the clothing 10 that is worn on the upper body of a patient. Accordingly, a patient may not feel inconvenience although the patient wears the human body correction apparatus 1 for a long time.

A zipper 20 may be installed in the middle of the front part of the clothing 10 from the top of the clothing to the bottom. A user can easily wear the human body correction apparatus 1 by undoing the zipper 20, passing his or her both arms through holes formed on the left and right sides of an upper part of the clothing 10, putting the clothing on his or her upper body, closing the zipper 20, and then wearing the clothing 10 on the upper body.

The curve compressor 100a may be disposed in the clothing 10. The curve compressor 100a may compress a curved part of a human body, that is, a target to be corrected. The curve compressor 100a may be connected to the intensity adjuster 100 through a wire 105.

The curve compressor 100a may basically include two parts. That is, the curve compressor 100a may include a first part that is fixed to the clothing 10 and a second part that is rotatably combined with the first part and that compresses the curved part by an operation of the second part being rotated by being pulled by the wire 105. The first part may be equipped with a shaft. The second part may be rotatably combined with the shaft.

The intensity adjuster 100 may be disposed in the clothing 10 by being spaced apart forward from the curve compressor 100a. The curve compressor 100a may be disposed on the rear side of the clothing 10 so that the curve compressor can compress the curved part. The intensity adjuster 100 may be disposed on the front side of the clothing 10 so that the intensity adjuster can be easily adjusted by a user. The intensity adjuster 100 may be connected to the curve compressor 100a through the wire 105. A user can adjust the compression intensity of the curve compressor 100a because the amount of winding and unwinding of the wire 105 connected to the intensity adjuster 100 is adjusted.

A guide rail 150 may be installed in the clothing 10. The guide rail 150 may provide guidance to up and down sliding movements of the curve compressor 100a and the intensity adjuster 100. The guide rail 150 may provide guidance to the up and down sliding movements of at least one of the curve compressor 100a and the intensity adjuster 100.

If the guide rail 150 provides guidance to the up and down sliding movements of the curve compressor 100a, the curved part can be corrected by adjusting up and down locations of the curve compressor 100a based on the location of the curved part of a patient because the up and down locations of the curve compressor 100a can be adjusted.

Furthermore, if the guide rail 150 provides guidance to the up and down sliding movements of the intensity adjuster 100, a patient can easily adjust the compression intensity of the curve compressor 100a by manipulating the intensity adjuster 100 for himself or herself because the up and down locations of the intensity adjuster 100 can be adjusted.

The guide rail 150 may move at least one of the curve compressor 100a and the intensity adjuster 100 from an arbitrary location of a human body to a specific location.

The guide rail 150 may include a pair of guide rails 150 that provides guidance to the up and down sliding movements of the curve compressor 100a, and may include a pair of guide rails 150 that provides guidance to the up and down sliding movements of the intensity adjuster 100. However, the guide rail 150 does not need to essentially include the pair of guide rails 150 in order to provide guidance to the up and down sliding movements of the curve compressor 100a, and may include at least one guide rail 150. Furthermore, the guide rail 150 does not need to essentially include the pair of guide rails 150 in order to provide guidance to the up and down sliding movements of the intensity adjuster 100, and may include at least one guide rail 150.

The curve compressor 100a, the intensity adjuster 100, and the guide rail 150 may be coupled to the clothing 10, and may be fixed and disposed in the clothing 10. However, in the present embodiment, only the guide rail 150 is coupled to the clothing 10 and fixed and disposed therein. The curve compressor 100a and the intensity adjuster 100 are disposed in the guide rail 150 in a way to be movable up and down.

The curve compressor 100a and the intensity adjuster 100 may be disposed in a portion corresponding to a left flank of a correction patient and a portion corresponding to a right flank of the correction patient, respectively, which are portions of the clothing 10 through which the curved part attributable to scoliosis will be corrected.

Two pairs of guide rails 150 that provide guidance to the up and down sliding movements of the curve compressor 100a and the intensity adjuster 100 that are disposed on the right flank side of the correction patient may be arranged on the right side of the clothing 10.

Furthermore, two pairs of guide rails 150 that provide guidance to the up and down sliding movements of the curve compressor 100a and the intensity adjuster 100 that are disposed on the left flank side of the correction patient may be arranged on the left side of the clothing 10.

Accordingly, a total of eight guide rails 150 that provide guidance to the up and down sliding movements of the left curve compressor 100a, the left intensity adjuster 100, the right curve compressor 100a, and the right intensity adjuster 100a may be installed in the clothing 10.

A Y-shaped compression band Y may be installed outside the clothing 10. The Y-shaped compression band Y may include a first end Y1, a second end Y2, and a third end Y3. The first end Y1 may extend to the left shoulder side in a back portion of the correction patient. The second end Y2 may extend from the first end Y1 so that the second end surrounds a right waist part in the back portion of the correction patient. The third end Y3 may be branched from a middle part of the first end Y1 and the second end Y2, and may extend to surround a left waist part in the back portion of the correction patient.

Furthermore, an L-shaped compression band L may be installed outside the clothing 10. The L-shaped compression band L may include a first end L1, a second end L2, and a third end L3. The first end L1 may extend to a right shoulder side in the back portion of the correction patient. The second end L2 may extend from the first end L1 to surround the left waist part of the correction patient. The third end L3 may extend from the second end L2 to surround the back portion and right waist of the correction patient.

Meanwhile, the curve compressor 100a is disposed to be closely attached to the outside of the human body of a correction patient, which corresponds to the curved part, that is, a portion that needs to be corrected, and may correct the curved part of the correction patient by applying predetermined correction pressure to the curved part.

More specifically, the intensity of the curve compressor 100a that is compressed to the outside of the human body of the correction patient when the amount of winding and unwinding of the wire 105 of the intensity adjuster 100 is adjusted may be adjusted. That is, when the wire 105 is wound by using the intensity adjuster 100, the curve compressor 100a may compress the outside of the human body of the correction patient with high pressure. In contrast, when the wire 105 is unwound by using the intensity adjuster 100, the curve compressor 100a may release compression that has been applied to the outside of the human body of the correction patient.

The intensity adjuster 100 is provided at a location that is spaced apart from the curve compressor 100a at a predetermined distance in the circumference direction of the clothing 10, and may perform a role to adjust compression intensity for the outside of the human body of the curve compressor 100a that is connected to the intensity adjuster through the wire 105.

The wearing type correction apparatus 1 for a human body according to an embodiment of the present disclosure may further include a pelvis band 160. The pelvis band 160 may be disposed at the bottom of the clothing 10. The pelvis band 160 may surround the pelvis of a patient by surrounding a portion of the clothing 10, which corresponds to the pelvis of the patient.

The pelvis band 160 may hold the pelvis of the patient when the curve compressor 100a compresses the curved part. Accordingly, efficiency of the curve compressor 100a that compresses the curved part can be improved.

A construction in which the curve compressor 100a and the intensity adjuster 100 that are installed to slide and move on the guide rail 150 up and down is described in detail. The curve compressor 100a and the intensity adjuster 100 perform different functions as described above, but may have a body 110, a support plate 140, and a rail moving block 145 to be described later as common components in order for the curve compressor and the intensity adjuster to be installed in the guide rail 150 in a way to slide and move up and down. However, unlike in the intensity adjuster 100, a dial adjuster 120 and a dial cover 130 to be described later may not be provided in the body 110 of the curve compressor 100a. In the following description, only a detailed construction of the intensity adjuster 100 is described, and a description of a detailed construction of the curve compressor 100a is omitted.

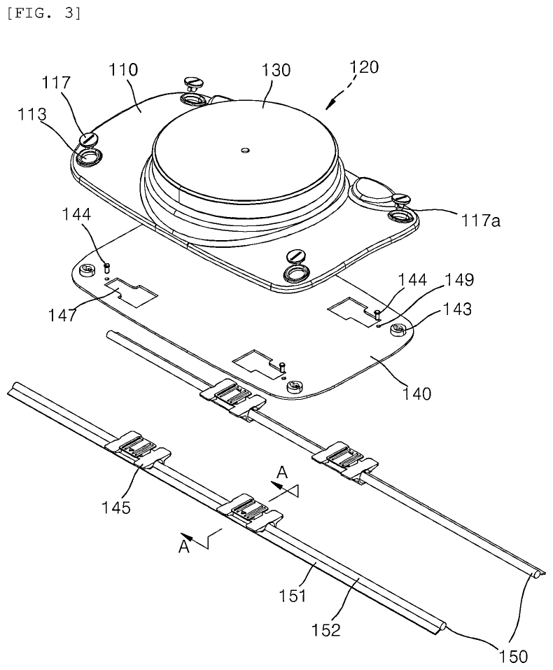

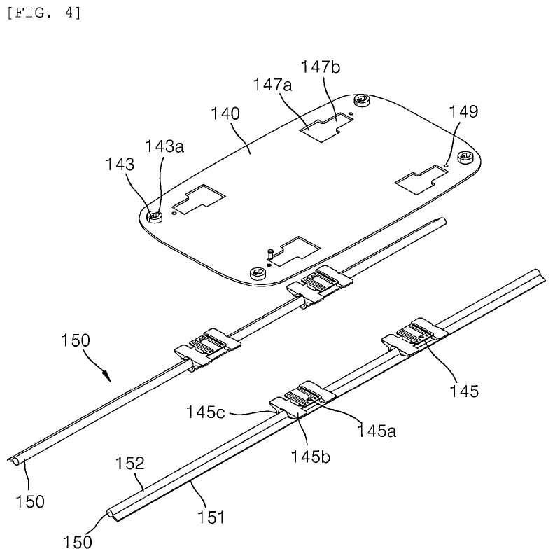

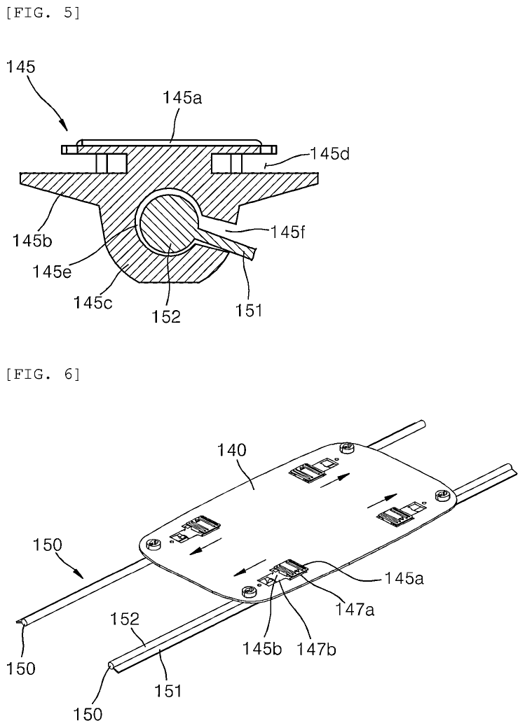

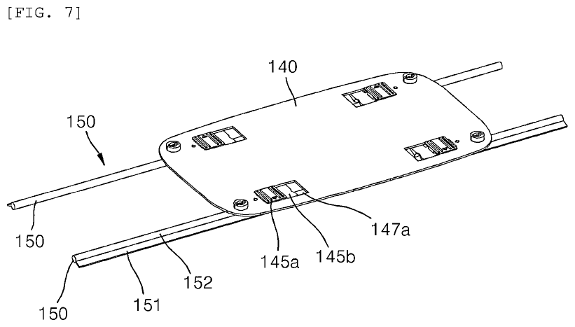

FIG. 3 is an exploded perspective view illustrating an installation structure of the intensity adjuster illustrated in FIGS. 1 and 2. FIG. 4 is an exploded perspective view except the body in FIG. 3. FIG. 5 is a cross-sectional view taken along line A-A of FIG. 3. FIGS. 6 and 7 are coupling sequence diagrams of the body and the support plate illustrated in FIG. 3.

Referring to FIGS. 3 to 7, the intensity adjuster 100 may include the body 110, the support plate 140, and the rail moving block 145.

The body 110 may form an appearance shape of the intensity adjuster 100. The body 110 is formed in a sheet form, and may be installed in an outer surface of the clothing 10. The support plate 140 may be disposed between the body 110 and the guide rail 150. The support plate 140 may be combined with the body 110. The rail moving block 145 may be combined with the support plate 140. The rail moving block 145 may be slidably combined with the guide rail 150 in the length direction of the guide rail 150.

A pair of rail moving blocks 145 may be installed in one guide rail 150. The number of rail moving blocks 145 that are installed in one guide rail 150 may be variously changed. At least one rail moving block 145 may be installed in one guide rail 150.

The body 110 may be formed in a sheet form in which the body has a width greater than separation distance between the pair of guide rails 150. Furthermore, the body 110 may be formed in a shape in which the body is generally convexed outward so that the body corresponds to the bending of the human body of a correction patient.

The intensity adjuster 100 may further include the dial adjuster 120. The dial adjuster 120 is rotatably combined with the body 110, and may adjust the amount of winding and unwinding of the wire 105.

Although not illustrated, the dial adjuster 120 may be rotatably provided in the outer part of the body 110. The wire 105 may be wound on or unwound from the outer circumferential surface of the dial adjuster 120.

The intensity adjuster 100 may further include the dial cover 130 that covers the dial adjuster 120. The dial cover 130 may be formed in a circular sheet form, may cover the dial adjuster 120 from the outside, and may rotate the dial adjuster 120 by an operation of the dial cover being rotated to one side or the other side.

The wire 105 that extends from the intensity adjuster 100 may be connected to one side of the curve compressor 100a. The curve compressor 100a may compress an appearance portion of the human body, that is, a portion to be corrected, when the wire 105 is wound on the outer circumferential surface of the dial adjuster 120 of the intensity adjuster 100. The compression of the curve compressor may be released when the wire 105 is unwound from the outer circumferential surface of the dial adjuster 120 of the intensity adjuster 100.

The body 110 may be fixed and installed in the support plate 140. The support plate 140 may be installed in the pair of guide rails 150 in the length direction of the guide rails 150 through the medium of the four rail moving blocks 145 in a way to be slidable and movable.

The support plate 140 may be disposed between the body 110 and the pair of guide rails 150. An outer surface of the support plate 140 may come into contact with an inner surface of the body 110. At least two rail moving blocks 145 may be disposed to protrude on an inner surface of the support plate 140. The support plate 140 may be combined with the pair of guide rails 150 in a way to be slidable and movable through the at least two rail moving blocks 145.

The intensity adjuster 100 may further include a holder fixing pin 117. The holder fixing pin 117 may combine the body 110 and the support plate 140. Corner parts of the body 110 and the support plate 140 may be mutually combined with each other by a plurality of holder fixing pins 117.

A through hole 113 may be formed in the body 110. One through hole 113 is formed at the corner part of the body 110, and a plurality of through holes 113 may be formed in the body 110. A boss part 143 that is inserted into the through hole 113 may be formed in the support plate 140. The boss part 143 may be formed on the outer surface of the support plate 140. The boss part 143 may be formed to protrude toward the body 110 on the outer surface of the support plate 140, which faces the body 110. The support plate 140 may be formed in the form of a quadrangle shape, that is, a shape corresponding to the body 110. One boss part 143 may be formed at the corner part of the support plate 140, and four boss parts 143 may be formed in the support plate 140.

The holder fixing pin 117 may be inserted into the boss part 143. That is, in the state in which the boss part 143 has been inserted into the through hole 113 of the body 110, a part of the holder fixing pin 117 except a head part of the holder fixing pin 117 may be inserted into a groove that is formed in the boss part 143 outside the body 110. In such a state, the head part of the holder fixing pin 117 may be locked in a protrusion on the internal wall of the through hole 113 of the body 110.

A locking protrusion 117a may be formed in the outer circumferential surface of the holder fixing pin 117. The locking protrusion 117a may be formed in the outer circumferential surface of the part of the holder fixing pin 117, which is inserted into the groove formed in the boss part 143.

Furthermore, a locking groove 143a that is fastened to the holder fixing pin 117 may be formed in the boss part 143. The locking groove 143a in which the locking protrusion 117a formed in the outer circumferential surface of the holder fixing pin 117 is locked may be formed within the groove of the boss part 143.

A worker can mutually combine the body 110 and the support plate 140 by inserting the holder fixing pin 117 into the boss part 143 and then rotating the holder fixing pin 117 to one side thereof so that the locking protrusion 117a of the holder fixing pin 117 is locked in the locking groove 143a of the boss part 143.

Two rail moving blocks 145 may be disposed in any one of the pair of guide rails 150, and may be connected to one side of the support plate 140 in the width direction thereof. Two rail moving blocks 145 may be disposed in the other of the pair of guide rails 150, and may be connected to the other side of the support plate 140 in the width direction thereof.

A plurality of mounting holes 147 may be formed in the support plate 140. The plurality of mounting holes 147 may include a first coupling hole 147a and a second coupling hole 147b. That is, the first coupling hole 147a and the second coupling hole 147b may be formed in the support plate 140. The second coupling hole 147b may extend from the first coupling hole 147a in the sliding direction of the rail moving block 145. The width of the second coupling hole 147b in a direction orthogonal to the sliding direction of the rail moving block 145 may be formed to be smaller than the width of the first coupling hole 147a. The rail moving block 145 may be fit into the first coupling hole 147a, may slide and move to the second coupling hole 147b, and may be combined with the support plate 140.

The rail moving block 145 may include a locking part 145a, a support part 145b, and a coupling part 145c.

The width of the locking part 145a in a direction orthogonal to the sliding direction of the rail moving block 145 may be formed to be equal to or smaller than the width of the first coupling hole 147a. Furthermore, the width of the locking part 145a in the direction orthogonal to the sliding direction of the rail moving block 145 may be formed to be greater than the width of the second coupling hole 147b.

The width of the support part 145b in a direction orthogonal to the sliding direction of the rail moving block 145 may be formed to be greater than each of the width of the first coupling hole 147a and the width of the second coupling hole 147b.

A locking groove 145d into which parts of the support plate 140, which are disposed on both sides of the second coupling hole 147b, are inserted may be formed between the locking part 145 and the support part 145b. That is, when a worker fits the locking part 145a of the rail moving block 145 into the first coupling hole 147a and the locking part 145a slides and moves to the second coupling hole 147b, the rail moving block 145 can be combined with the support plate 140 because the locking part 145a is locked in the locking groove 145d, that is, the parts of the support plate 140, which are disposed on both sides of the second coupling hole 147b.

In the state in which the rail moving block 145 has been combined with the support plate 140 as described above, one surface of the locking part 145a may come into contact with the outer surface of the support plate 140, and one surface of the support part 145b may come into contact with the inner surface of the support plate 140. In this case, the one surface of the locking part 145a and the one surface of the support part 145b may be surfaces that face each other. Specifically, the one surface of the locking part 145a may be an inner surface of the locking part 145a, and the one surface of the support part 145b may be an outer surface of the support part 145b.

The first coupling hole 147a and the second coupling hole 147b may be formed at multiple places of the support plate 140. The first coupling hole 147a and the second coupling hole 147b are holes that are mutually connected. The length and width of the first coupling hole 147a may be formed to be greater than the length and width of the locking part 145a of the rail moving block 145, but the length and width of the second coupling hole 147b may be formed to be smaller than the length and width of the locking part 145a of the rail moving block 145. Accordingly, the locking part 145a of the rail moving block 145 may penetrate the first coupling hole 147a, but cannot penetrate the second coupling hole 147b.

Furthermore, the length and width of the support part 145b of the rail moving block 145 may be formed to be greater than the length and width of each of the first coupling hole 147a and second coupling hole 147b of the support plate 140. Accordingly, the support part 145b of the rail moving block 145 cannot penetrate both the first coupling hole 147a having a relatively large hole and the second coupling hole 147b having a relatively small hole, and may be supported by the inner surface of the support plate 140.

The locking part 145a and support part 145b of the rail moving block 145 are connected by a mutual connection part (a reference numeral not indicated). It is preferred that the sizes of the length and width of the connection part are formed to be smaller than the sizes of the length and width of the second coupling hole 147b so that at least the rail moving block 145 can slide and move toward the second coupling hole 147b.

Accordingly, in the state in which the locking part 145a of the rail moving block 145 penetrates the first coupling hole 147a of the support plate 140 and the support part 145b of the rail moving block 145 has been supported by the inner surface of the support plate 140, when the rail moving block 145 is moved toward the second coupling hole 147b and the fixing pin 144 is then fixed to the fixing hole 149 formed in the support plate 140 through the fixing hole 149, a process of combining the rail moving block 145 with the support plate 140 is completed. In an embodiment of the present disclosure, the first coupling hole 147a and the second coupling hole 147b may be formed at each of the four places of one support plate 140, and each of the four rail moving blocks 145 may be combined with the first coupling hole 147a and the second coupling hole 147b.

The coupling part 145c may be formed to protrude on the other surface of the support part 145b. In this case, the other surface of the support part 145b may be an inner surface of the support part 145b. A guide groove 145e that is slidably combined with the guide rail 150 may be formed in the coupling part 145c.

The guide rail 150 may include an attachment plate 151 and a guide protrusion 152.

The attachment plate 151 may be combined with the clothing 110. The attachment plate 151 may be combined with the clothing 110 by at least one of sewing and bonding methods.

The guide protrusion 152 may be formed on one side of the attachment plate 151 in a direction orthogonal to the sliding direction of the rail moving block 145. The guide protrusion 152 may be rotatably inserted and disposed in the guide groove 145e formed in the coupling part 145c of the rail moving block 145.

The attachment plate 151 is firmly fixed to the outer surface of the clothing 10, and the guide protrusion may be freely rotatably fixed on the attachment point of the attachment plate 151.

The guide groove 145e formed in the coupling part 145c of the rail moving block 145 and the guide protrusion 152 formed in the guide rail 150 are each formed to have a circular cross section. In the state in which the guide protrusion 152 has been inserted into the guide groove 145e and the rail moving block 145 has been installed in the guide rail 150 in a way to be slidable and movable up and down, the rail moving block 145 can be smoothly slid and moved up and down.

An opening 145f through which one side of the guide groove 145e is opened may be formed in the coupling part 145c of the rail moving block 145. In the state in which the guide protrusion 152 of the guide rail 150 has been inserted into the guide groove 145e, the attachment plate 151 of the guide rail 150 may be disposed in the opening 145f.

The guide protrusion 152 can be prevented from falling outside within the guide groove 145e because the opening width of the opening 145f is formed to be smaller than the diameter of the guide groove 145e. Furthermore, the rail moving block 145 can be smoothly slid and moved up and down because the opening width of the opening 145f is formed to be greater than the thickness of the attachment plate 151.

The diameter of the guide protrusion 152 may be formed to be greater than the thickness of the attachment plate 151. The diameter of the guide protrusion 152 may be formed to be smaller than the diameter of the guide groove 145e formed in the coupling part 145c of the rail moving block 145.

A cross-sectional shape of the coupling part 145c of the rail moving block 145 may be an approximately circular shape. The inner diameter of the coupling part 145c is formed to be greater than the outer diameter of the guide protrusion 152 of the guide rail 150. When the curve compressor 100a or the intensity adjuster 100 slides and moves up and down, the rail moving block 145 can be easily slid and moved up and down along the guide protrusion 152 without being locked in the guide protrusion 152.

The wearing type correction apparatus 1 for a human body constructed as above according to an embodiment of the present disclosure is disposed in the clothing 10 that is worn on an upper body of a correction patient, and is provided to compress a part of the human body of the correction patient who suffers from a scoliosis symptom. In this case, the wearing type correction apparatus for a human body may be provided in a way that at least one of the curve compressor 100a and the intensity adjuster 100 can slide and move along the guide rail 150 so that a compression portion is changed from any one point outside the human body to a specific point.

However, the wearing type correction apparatus 1 for a human body according to an embodiment of the present disclosure is not essentially limited to the clothing 10 that is worn on the upper body of a correction patient, and may have its versatility expanded by being applied to clothing or equipment that is worn on various portions of a human body.

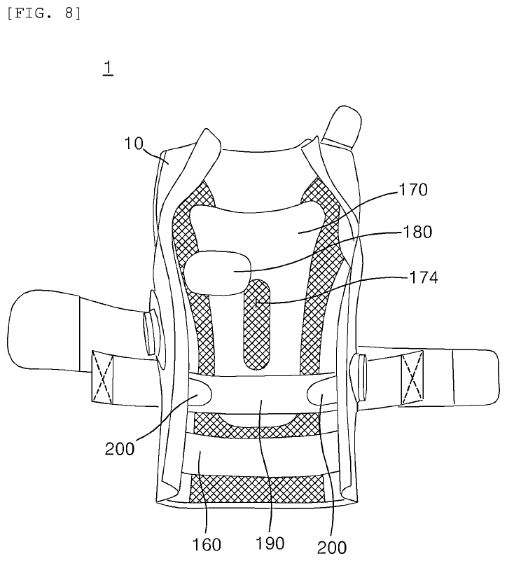

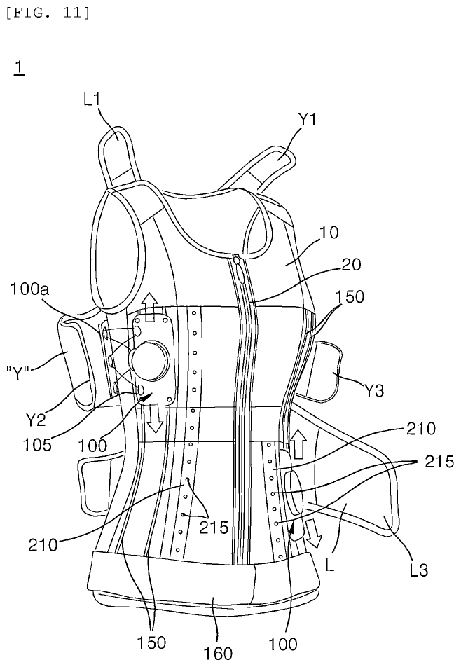

The wearing type correction apparatus 1 for a human body according to an embodiment of the present disclosure further includes components for improving efficiency of the curve compressor 100a that compresses the curved part. This is described below with reference to FIGS. 8 to 10.

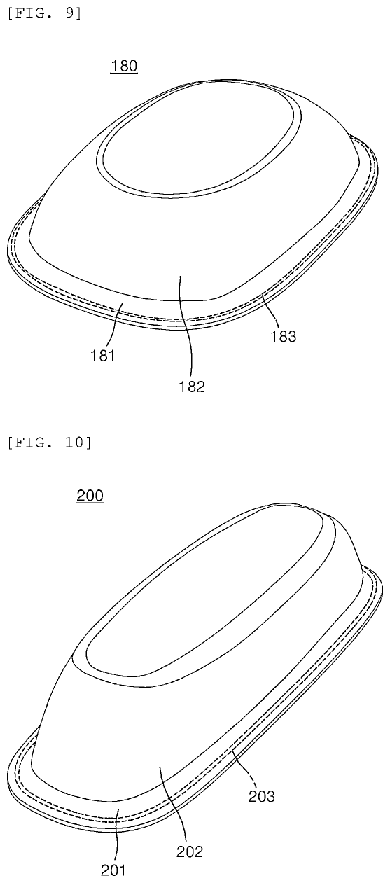

FIG. 8 is a diagram illustrating the inner surface of clothing illustrated in FIG. 1. FIG. 9 is a perspective view illustrating a hump pad illustrated in FIG. 8. FIG. is a perspective view illustrating a flank pad illustrated in FIG. 8.

Referring to FIGS. 8 to 10, the wearing type correction apparatus 1 for a human body according to an embodiment of the present disclosure may include the pelvis band 160, a back reinforcement plate 170, a hump pad 180, a flank band 190, and a flank pad 200.

The pelvis band 160 may be disposed at the bottom of the clothing 10. A part of the pelvis band 160 may be disposed on the inside of the clothing 10. The remaining part of the pelvis band 160 may be disposed outside the clothing 10. The pelvis band 160 may surround the pelvis of a patient by surrounding a part of the clothing 10, which corresponds to the pelvis of the patient. The pelvis band 160 may be formed of an elastic material capable of elastically compressing the pelvis of the patient.

When the curve compressor 100a compresses the curved part, the pelvis band 160 can hold the pelvis of the patient. Accordingly, efficiency of the curve compressor 100a that compresses the curved part can be improved.

The back reinforcement plate 170 may be disposed on an inner surface of a back part of the clothing 10. The back reinforcement plate 170 is made of a material that is stiffer than the material of the back part of the clothing 10. When the curve compressor 100a compresses the curved part, the back reinforcement plate firmly supports the back part of a patient, so that efficiency of the curve compressor 100a that compresses the curved part can be improved.

An opening hole 174 that is long up and down may be formed in the back reinforcement plate 170. The opening hole 174 may be formed in the middle between the left and right sides of the back reinforcement plate 170. The opening hole 174 may be formed at a location corresponding to the backbone of a patient. In the state in which a patient has worn the human body correction apparatus 1, a bent backbone of the patient can pass through the opening hole 174 without coming into contact with the back reinforcement plate 170 of the stiff material. Accordingly, the patient may not feel inconvenient due to the wearing of the human body correction apparatus 1.

The hump pad 180 is attached to an arbitrary location related to a location of the back reinforcement plate 174 at which the curve compressor 100a compresses the curved part, and may compress a hump part of a human body. In this case, the arbitrary location may be a location that is arbitrarily selected by a patient or a doctor.

The hump pad 180 may include a base 181 and a cushion protrusion 182. The base 181 may be formed in a sheet form. The base 181 may be attached to the back reinforcement plate 170. The cushion protrusion 182 may be formed to protrude on one surface of the base 181. The cushion protrusion 182 may be formed of a material having cushion power capable of compressing a human body. The base 181 may be formed of a stiff material capable of supporting the cushion protrusion 182. Velcro may be attached to the other surface of the base 181. The hump pad 180 may be attached to the arbitrary location of the back reinforcement plate 170 through the Velcro.

Meanwhile, a wire 183 of a metal material may be disposed within the edge of the base 181. After the human body correction apparatus 1 is worn and an X-ray photo thereof is photographed in the state in which the hump pad 180 has been attached to the back reinforcement plate 170, the wire 183 may appear white in the photographed X-ray photo because the wire 183 is formed of the metal material. Accordingly, a doctor can easily identify the location and state of the hump part by monitoring the wire 183 in the X-ray photo.

The flank band 190 may be disposed in the clothing 10. A part of the flank band 190 may be disposed inside the clothing 10, and the remaining part of the flank band 190 may be disposed outside the clothing 10. The flank band 190 may surround a part of the clothing 10, which corresponds to the flank of a patient. The flank band 190 may be formed of an elastic material capable of elastically compressing the flank of the patient.

When the curve compressor 100a compresses the curved part, the flank band 190 can hold the flank of the patient. Accordingly, efficiency of the curve compressor 100a that compresses the curved part can be improved.

The flank pad 200 is attached to an arbitrary location related to a location of the flank band 190 at which the curve compressor 100a compresses the curved part, and may compress a human body. In this case, the arbitrary location may be a location that is arbitrarily selected by a patient or a doctor.

The flank pad 200 may be formed to have the same construction as the hump pad 180. However, the flank pad 200 may be formed to have a longer length than the hump pad 180. The flank pad 200 has the same construction as the hump pad 180 except only its shape, and may include a base 201 and a cushion protrusion 202.

The base 201 may be formed in a sheet form. The base 201 may be attached to the flank band 190. The cushion protrusion 202 may be formed to protrude on one surface of the base 201. The cushion protrusion 202 may be formed of a material having cushion power that compresses a human body. The base 201 may be formed of a stiff material capable of supporting the cushion protrusion 202. Velcro may be attached to the other surface of the base 201. The flank pad 200 may be attached to the arbitrary location of the flank band 190 through the Velcro.

Meanwhile, a wire 203 of a metal material may be disposed within the edge of the base 201. After the human body correction apparatus 1 is worn and an X-ray photo thereof is photographed in the state in which the flank pad 200 has been attached to the flank band 190, the wire 203 may appear white in the photographed X-ray photo because the wire 203 is formed of the metal material. Accordingly, a doctor can easily identify the locations and states of the curved part and the hump part by monitoring the wire 203 in the X-ray photo.

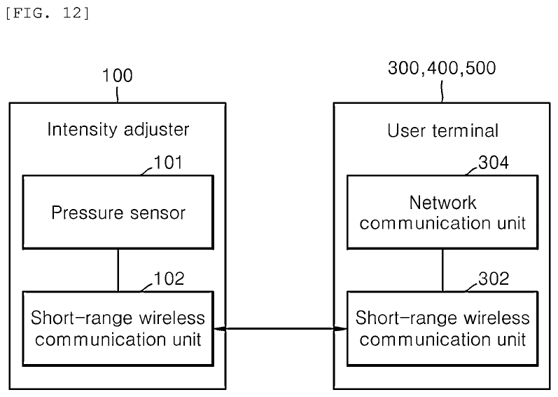

A doctor can easily identify the locations and states of the curved part and the hump part by monitoring the wires 183 and 203 in the X-ray photo because the wires 183 and 203 of the metal material are disposed within the hump pad 180 and the flank pad 200 as described above. In order for the locations and states of the curved part and the hump part to be more specifically identified in the X-ray photo, the wearing type correction apparatus 1 for a human body according to an embodiment of the present disclosure may further include a point band 210 to be described later. The point band 210 is described below with reference to FIG. 11.

FIG. 11 is a diagram illustrating the state in which the point band has been attached to FIG. 1.

Referring to FIG. 11, the point band 210 may be attached to the clothing 10. The point band 210 may be attached to an arbitrary location of the clothing 10. In this case, the arbitrary location may be a location that is arbitrarily selected by a doctor.

A plurality of metal points 215 that is spaced apart from each other up and down may be disposed in the point band 210. After the human body correction apparatus 1 is worn and an X-ray photo thereof is photographed in the state in which the point band 210 has been attached to the clothing 10, the metal points 215 may appear white in the photographed X-ray photo because the metal point 215 is formed of the metal material. A doctor can specifically identify the locations and states of the curved part and the hump part by monitoring the metal points 215 in the X-ray photo.

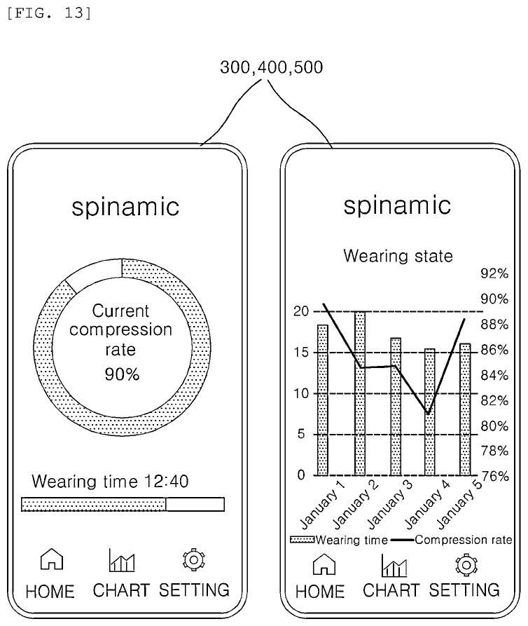

FIG. 12 is a control block diagram of the wearing type correction apparatus for a human body according to an embodiment of the present disclosure.

Referring to FIG. 12, a pressure sensor 101 may be installed in the intensity adjuster 100. The pressure sensor 101 does not need to be essentially installed only in the intensity adjuster 100. That is, the pressure sensor 101 may be installed in at least one of the curve compressor 100a and the intensity adjuster 100, that is, components the locations of which are adjusted up and down. The pressure sensor 101 may be installed in at least one of the Y-shaped compression band Y, the L-shaped compression band L, the pelvis band 160, and the flank band 190. However, if the pressure sensor 101 is installed in at least one of the curve compressor 100a and the intensity adjuster 100, that is, components the locations of which are adjusted up and down as in the present embodiment, there is an advantage in that the compression intensity can be accurately detected at a location closest to the curved part, that is, a target to be corrected, by adjusting the up and down locations of the curve compressor 100a and the intensity adjuster 100 depending on the location of the curved part of a human body, that is, a target to be corrected.

The pressure sensor 101 may include all of sensors capable of detecting the compression intensity of the curve compressor 100a. For example, the pressure sensor 101 may be a stain gauge pressure sensor, an electrostatic capacitive pressure sensor, or a piezoelectric pressure sensor.

A short-range wireless communication unit 102 may be further installed in the intensity adjuster 100. The short-range wireless communication unit 102 does not need to be essentially installed only in the intensity adjuster 100. That is, the short-range wireless communication unit 102 may be installed in at least one of the curve compressor 100a and the intensity adjuster 100, that is, components the locations of which are adjusted up and down. The short-range wireless communication unit may be mounted on a printed circuit board along with the pressure sensor 101.

A scoliosis patient includes a left scoliosis patient or a right scoliosis patient. It is preferred that the pressure sensor 101 and the short-range wireless communication unit 102 are installed at locations at which both the left scoliosis patient and the right scoliosis patient can be measured. To this end, the curve compressor 100a and the intensity adjuster 100 may be installed on the right and left sides on the basis of the central part of the clothing 10, respectively. The pressure sensor 101 and the short-range wireless communication unit 102 may be installed in the curve compressor 100a and the intensity adjuster 100 that are installed on the left side on the basis of the central part of the clothing 10, and in the curve compressor 100a and the intensity adjuster 100 that are installed on the right side on the basis of the central part of the clothing 10.

The short-range wireless communication unit 102 may communicate with a user terminal 300, 400, 500 in which a correction management service app for informing a user of data including compression intensity detected by the pressure sensor 101 has been installed. It is preferred that the user terminal 300, 400, 500 is equipped with a short-range wireless communication unit 302 that performs short-range wireless communication with the short-range wireless communication unit 102. In this case, the short-range wireless communication unit 102, 302 may be a Bluetooth communication unit or a Wi-Fi communication unit.

The correction management service app installed in the user terminal 300, 400, 500 may calculate data including compression intensity detected by the pressure sensor 101 and show the data to a user. In this case, a business operator who manufactures and sells the wearing type correction apparatus 1 for a human body according to an embodiment of the present disclosure may provide the correction management service app to a user. The data including the compression intensity, which are shown to a user through the correction management service app, may include the compression intensity (a compression rate) of the curve compressor 100a, a wearing time of the human body correction apparatus 1 that is calculated by using the compression intensity, compliance of a correction patient that is calculated by using the compression intensity and the wearing time, etc.

That is, the correction management service app installed in the user terminal 300, 400, 500 may output the compression intensity detected by the pressure sensor 101 so that a user can view the compression intensity, may calculate the time for which the compression intensity detected by the pressure sensor 101 is maintained as the wearing time of the human body correction apparatus 1 and then output the wearing time so that the user can view the wearing time, and may calculate the compliance of the user by using the compression intensity and the wearing time and output the compliance so that the user can view the compliance.

The user terminal 300, 400, 500 may include all of devices each having the correction management service app installed therein and each including a display unit that shows, to a user, data including compression intensity, a wearing time, and compliance that are output to the correction management service app. For example, the user terminal 300, 400, 500 may be a mobile phone or a table PC.

The compression intensity, the wearing time, and the compliance that are output from the correction management service app may be output in the form of graphs or numbers. A user can check the compression intensity of the correction management service app, which is displayed on a screen of the user terminal 300, 400, 500.

Furthermore, whether a scoliosis patient observes a wearing time (e.g., 18 hours or more) of the wearing type correction apparatus 1 for a human body of the present disclosure and whether compression intensity is correctly maintained are important. A user can easily check whether the wearing time of the human body correction apparatus 1 is observed and whether compression intensity is correctly maintained through the correction management service app installed in the user terminal 300, 400, 500 because the pressure sensor 101 and the short-range wireless communication unit 102 are included in the wearing type correction apparatus 1 for a human body of the present disclosure.

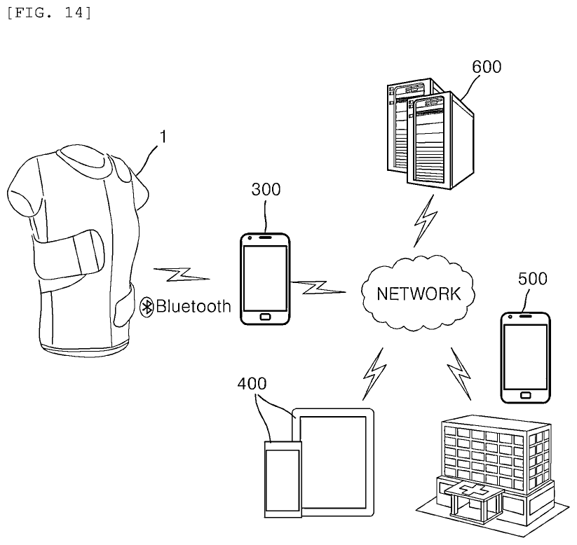

FIG. 13 is a diagram illustrating the state in which the correction management service app installed in the user terminal illustrated in FIG. 12 is displayed on a screen of the user terminal.

Referring to FIG. 13, when the correction management service app installed in the user terminal 300, 400, 500 is executed, a home icon, a chart icon, and a setting icon may be displayed at the bottom of a screen of the user terminal 300, 400, 500. The chart icon may be displayed in the middle of the bottom of a screen of the user terminal 300, 400, 500. The home icon may be displayed at a location spaced apart from the chart icon on the left side thereof. The setting icon may be displayed at a location spaced apart from the chart icon on the right side thereof. Letters “HOME” may be displayed below the home icon. Letters “CHART” may be displayed below the chart icon. Letters “SETTING” may be displayed below the setting icon.

When a user touches the home icon, a screen of the user terminal 300, 400, 500, such as that illustrated on the left side of FIG. 13, may be displayed as a home screen. When a user touches the chart icon, a screen of the user terminal 300, 400, 500, such as that illustrated on the right side of FIG. 13, may be displayed as a chart screen. Furthermore, when a user touches the setting icon, a screen of the user terminal 300, 400, 500 may be displayed as a setting screen. A user may input the wearing time and compression intensity (compression rate) of the human body correction apparatus 1, which have been prescribed by a doctor, to the setting screen that is displayed after the setting icon is touched.

As illustrated in the left figure of FIG. 13, compression intensity that is input on the setting screen versus a current compression rate may be displayed on the home screen along with a circular graph. A current wearing time to a wearing time that is input on the setting screen may be displayed on the home screen along with a straight type graph that is long left and right.

As illustrated in the right figure of FIG. 13, a daily wearing time to a wearing time that is input on the setting screen may be displayed on the chart screen along with a straight type graph that is long up and down. Compression intensity that is input on the setting screen versus a daily compression rate may be displayed on the chart screen in the form of a continuous straight type graph according to a flow of time.

FIG. 14 is communication state diagram of the wearing type correction apparatus for a human body according to an embodiment of the present disclosure.

Referring to FIGS. 12 and 14, the user terminal 300, 400, 500 may further include a network communication unit 304. The user terminal 300, 400, 500 may communicate with a server 600 through the network communication unit 304.

Furthermore, the user terminal 300, 400, 500 may include a terminal 300 for a patient, a terminal 400 for a guardian, and a terminal 500 for a doctor. The terminal 300 for a patient may be possessed by a patient. The terminal 400 for a guardian may be possessed by a guardian of a patient. The terminal 500 for a doctor may be possessed by a doctor in charge of an outpatient in a hospital.

The short-range wireless communication unit 302 of the terminal 300 for a patient may receive data including compression intensity detected by the pressure sensor 101 of the wearing type correction apparatus 1 for a human body, through communication with the short-range wireless communication unit 102 included in the wearing type correction apparatus 1 for a human body.

Furthermore, the network communication unit 304 of the terminal 300 for a patient may transmit the received data including the compression intensity to the server 600 through communication with the server 600 over a network communication network. The server 600 may store the data including the compression intensity, which are received from the terminal 300 for a patient.

Furthermore, the network communication unit 304 of each of the terminal 400 for a guardian and the terminal 300 for a doctor may receive data including compression intensity, which are stored in the server 600, from the server 600, through communication with the server 600 over a network communication network.

As described above, data including compression intensity detected by the pressure sensor 101 of the wearing type correction apparatus 1 for a human body may be shared with the terminal 300 for a patient, the terminal 400 for a guardian, and the terminal 500 for a doctor.

Hereinafter, a method of monitoring corrections using the wearing type correction apparatus 1 for a human body is described in detail. In describing the method of monitoring corrections according to an embodiment of the present disclosure, contents that are redundant with the aforementioned contents will be mentioned in brief or a description thereof may be omitted.

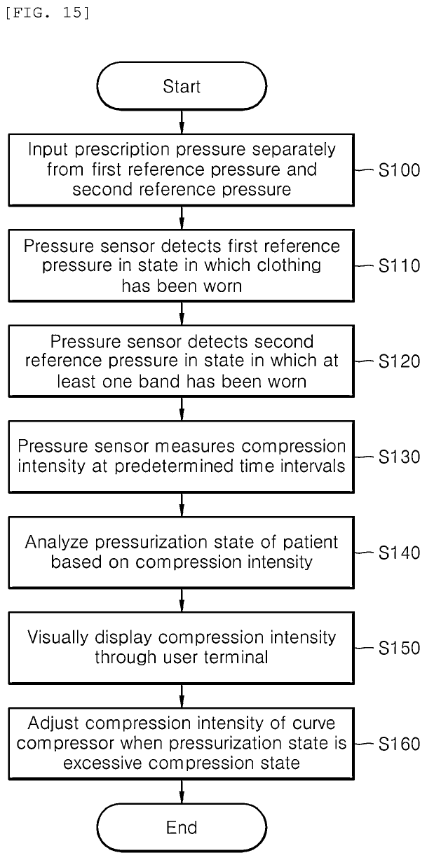

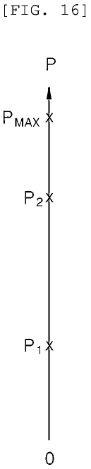

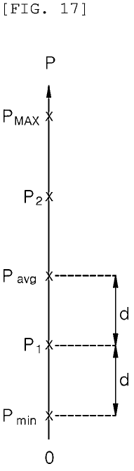

FIG. 15 is an exemplary flowchart of the method of monitoring corrections using a wearing type correction apparatus for a human body according to an embodiment of the present disclosure. FIG. 16 is a diagram for describing a process of detecting first reference pressure and second reference pressure in the method of monitoring corrections according to FIG. 15. FIG. 17 is a diagram for describing average pressure and minimum pressure in the method of monitoring corrections according to FIG. 15.

Referring to FIGS. 1, 12, and 15 to 17, the method of monitoring corrections using a wearing type correction apparatus for a human body according to an embodiment of the present disclosure may include a clothing wearing detection step S110, a band wearing detection step S120, a compression intensity detection step S130, and a display step S150.

In the clothing wearing detection step S110, a patient may wear the clothing 10 of the wearing type correction apparatus 1 for a human body. In the state in which the patient has worn the clothing 10, the pressure sensor 101 installed in at least one of the curve compressor 100a and the intensity adjuster 100 that are disposed in the clothing 10 may detect first reference pressure P1. A pressurization state described later may be analyzed based on the first reference pressure P1. Accordingly, whether the patient wears the clothing 10 of the wearing type correction apparatus 1 for a human body normally may be identified.

In the compression intensity detection step S120, the patient may wear at least one band that is disposed in the clothing of the wearing type correction apparatus 1 for a human body. Illustratively, the band may include at least one of the L-shaped compression band L, the Y-shaped compression band Y, the pelvis band 160, the flank band 190, and the point band 210, that is, components of the wearing type correction apparatus 1 for a human body according to an embodiment of the present disclosure.

When the patient wears at least one band, the human body of the patient may be pressurized compared to a case in which the patient wears only the clothing 10. In this case, in the compression intensity detection step S120, in the state in which the patient has worn at least one band disposed in the clothing 10, the pressure sensor 101 may detect second reference pressure P2. A pressurization state described later may be analyzed based on the second reference pressure P2. Accordingly, whether the patient wears at least one band L, Y, 160, 190, and 210 of the wearing type correction apparatus 1 for a human body normally may be identified.

Furthermore, the method of monitoring corrections using a wearing type correction apparatus for a human body according to an embodiment of the present disclosure may include the compression intensity detection step S130. In the compression intensity detection step S130, the pressure sensor 101 may measure compression intensity at predetermined time intervals. In this case, the compression intensity detection step S130 may be performed after the first reference pressure P1 is detected in the clothing wearing detection step S110 and the second reference pressure P2 is detected in the band wearing detection step S120.

In the compression intensity detection step S130, in general, the pressure sensor 101 may enter an operation mode at constant time intervals (e.g., 1 minute to 5 minutes) from a sleep mode in which consumption power has been minimized. In the operation mode, the pressure sensor 101 may measure compression intensity that is applied to the human body of the patient. Illustratively, the compression intensity may be measured in a kgf unit, but is not essentially limited thereto.

When the pressure sensor 101 measures the compression intensity, the pressure sensor 101 may transmit the measured compression intensity through short-range communication with at least one of the user terminals 300, 400, and 500 that is wirelessly connected thereto. Illustratively, the pressure sensor 101 may transmit the measured compression intensity to the terminal 300 for a patient through short-range communication. The terminal 300 for a patient may transmit the measured compression intensity to at least one of the terminal 400 for a guardian, the terminal 500 for a doctor, and the server 600 over a network. When the compression intensity is transmitted and a predetermined wait time (e.g., 10 seconds) elapses, the measured compression intensity may be stored in memory embedded in the pressure sensor 101. The pressure sensor 101 may switch to the sleep mode again.

If the pressure sensor 101 is not connected to any one of the user terminals 300, 400, and 500 and the server 600, the measured compression intensity may be stored in the memory embedded in the pressure sensor 101, and the pressure sensor 101 may switch to the sleep mode again.

The method of monitoring corrections using a wearing type correction apparatus for a human body according to an embodiment of the present disclosure may include the display step S150. In the display step S150, the user terminal 300, 400, 500 may visually display the compression intensity measured in the compression intensity detection step S130. Illustratively, the user terminal 300, 400, 500 may display the compression intensity in the form of a circular graph. As another example, the user terminal 300, 400, 500 may display the compression intensity in the form of a broken-line graph. When the compression intensity is displayed in the display step S150, the compression intensity may be displayed as average compression intensity for each time, but is not essentially limited to the listed examples.

As described above, a user (at least one of a patient, a guardian, and a doctor) may identify whether the patient wears the wearing type correction apparatus 1 for a human body correctly and spine corrections are smoothly performed through the user terminals 300, 400, and 500 that are possessed by the patient, the guardian, and the doctor, respectively.

Furthermore, the method of monitoring corrections using a wearing type correction apparatus for a human body according to an embodiment of the present disclosure may further include a pressurization state analysis step S140. In the pressurization state analysis step S140, the pressurization state of the patient may be analyzed based on the measured compression intensity. That is, whether the human body of the patient is corrected normally may be analyzed through the measured compression intensity. The pressurization state analysis step S140 may be performed by at least one operation of the user terminals 300, 400, and 500 and the server 600 that communicates with the user terminals 300, 400, and 500.

Hereinafter, the range of a pressurization state in which the measured compression intensity is classified in the pressurization state analysis step S140 is described in detail.

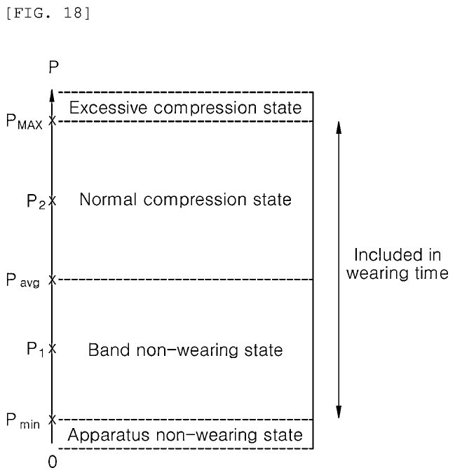

FIG. 18 is a diagram illustrating a pressurization state that is analyzed based on measured compression intensity.

Referring to FIGS. 16 to 18, the pressurization state may include at least three states based on the first reference pressure P1 and the second reference pressure P2. Illustratively, the pressurization state may include an apparatus non-wearing state in which a patient has not worn the clothing 10, a band non-wearing state in which a patient has worn the clothing 10, but has not worn the band, a normal wearing state in which a patient has worn the clothing 10 and the band, and an excessive compression state in which a patient has worn the clothing 10 and the band, but compression intensity has been excessively applied to the human body of the patient.

The states may be divided based on the first reference pressure P1 and the second reference pressure P2. Illustratively, the excessive compression state may be a state having the compression intensity of maximum pressure PMAX or more that is free of a pain for a patient. That is, when the compression intensity of the maximum pressure PMAX or more is applied to the human body of the patient, the patient may experience inconvenience, such as a pain (e.g., thoracalgia) or a respiratory difficulty attributable to pressurization. Illustratively, the maximum pressure PMAX may be 5 kgf, but is not essentially limited to the disclosed value.

Furthermore, the normal wearing state may be a state in which measured compression intensity P is average pressure (Pavg; 0.5*(P1+P2)) or more, that is, an average value of the first reference pressure P1 and the second reference pressure P2, and is less than the maximum pressure PMAX. When the compression intensity is within the range of the normal wearing state, it may be determined that a patient accurately wears the wearing type correction apparatus 1 for a human body for posture corrections.

Furthermore, the band non-wearing state may be a state in which the measured compression intensity P is minimum pressure Pmin or more and is less than the average pressure Pavg. In this case, the minimum pressure Pmin may be pressure (Pavg−d) that is obtained by subtracting a deviation d between the first reference pressure P1 and the average pressure Pavg from the first reference pressure P1. That is, the minimum pressure Pmin may be (1.5*P1−0.5*P2). When the compression intensity P is within the range of the band non-wearing state, it may be determined that a patient has worn the clothing 10 of the wearing type correction apparatus 1 for a human body, but has not worn at least one band disposed in the clothing 10 normally.

Furthermore, the apparatus non-wearing state may be a state in which the measured compression intensity P is less than the minimum pressure Pmin. When the compression intensity P is within the range of the apparatus non-wearing state, it may be determined that a patient has not worn the clothing 10 of the wearing type correction apparatus 1 for a human body normally. That is, if a pressurization state is determined as the apparatus non-wearing state, it may be determined that a patient has not worn the clothing 10 or has not fastened the zipper 20 of the clothing 10.

Whether the human body of a patient is corrected normally can be accurately analyzed by analyzing the pressurization state of the patient through the pressurization state analysis step S130 as described above.

Hereinafter, in another example of the method of monitoring corrections using a wearing type correction apparatus for a human body according to an embodiment of the present disclosure, contents in which a different range criterion for the pressurization state applied in the pressurization state analysis step S140 as prescription pressure is input is applied are described.

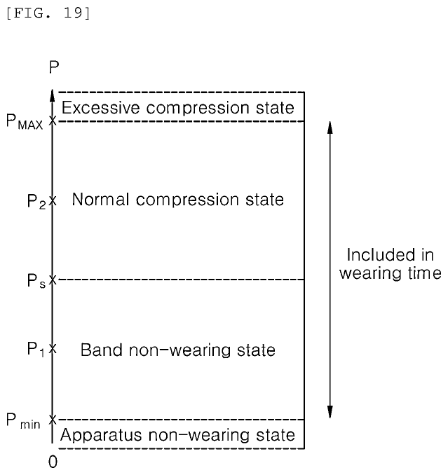

FIG. 19 is a diagram illustrating a pressurization state that is analyzed based on compression intensity measured through prescription pressure.

Referring to both FIGS. 15 and 19, the method of monitoring corrections using a wearing type correction apparatus for a human body according to an embodiment of the present disclosure may further include a prescription pressure input step S100. The prescription pressure input step S100 may be performed prior to the pressurization state analysis step S140. Illustratively, the prescription pressure input step S100 may be performed prior to the clothing wearing detection step S110, and may be performed prior to the band wearing detection step S120. Furthermore, the prescription pressure input step S100 may be performed prior to the compression intensity detection step S130.

In the prescription pressure input step S100, a user may input some information of a patient through the correction management service app that is displayed on the user terminal 300, 400, 500. Illustratively, the user may input at least one of prescription pressure Ps and a prescription time that are necessary to correct the human body of a patient through the correction management service app. The prescription pressure Ps may be recommended compression intensity for a patient, which is prescribed by a doctor through treatment. The user terminal may receive the prescription pressure Ps separately from the first reference pressure P1 and the second reference pressure P2 that are detected by the pressure sensor 101. A pressurization state may be classified based on the prescription pressure Ps.

More specifically, the pressurization state may include an excessive compression state, a normal compression state, a band non-wearing state, and an apparatus non-wearing state, such as those described above. In this case, the range of each of the excessive compression state and the apparatus non-wearing state may be the same as that described above. However, the normal compression state may be a state in which the measured compression intensity P is the prescription pressure Ps or more and is less than the maximum pressure PMAX. The band non-wearing state may be a state in which the measured compression intensity P is the minimum pressure Pmin or more and is less than the prescription pressure Ps. Accordingly, personalization and optimized monitoring can be performed on a patient and more suitable human body corrections can be performed on the patient because the pressurization state is classified and analyzed based on the prescription pressure Ps.

Hereinafter, a process of displaying the wearing time of the wearing type correction apparatus for a human body is described.

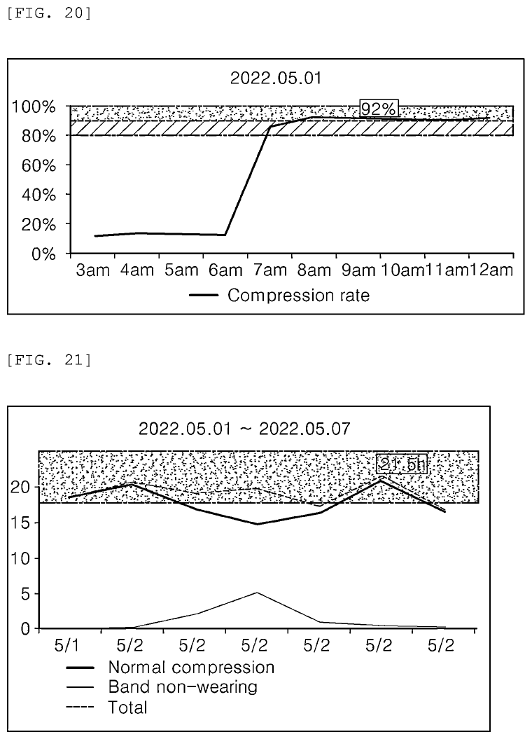

FIG. 20 is a diagram illustrating a daily compression rate graph that is displayed on the user terminal. FIG. 21 is a diagram illustrating a daily wearing time graph that is displayed on the user terminal.

Referring to FIGS. 15 and 18 to 21, in the display step S150, a wearing time that is calculated by using the compression intensity P may also be displayed on the user terminal 300, 400, 500. The wearing time may be calculated by adding the time for which a patient has worn the wearing type correction apparatus 1 for a human body. As illustrated in FIGS. 17 and 18, the wearing time may be calculated by adding the time for which the pressurization state is the normal compression state and the band non-wearing state. The wearing time may be calculated by further adding the time for which the pressurization state is, that is, the excessive compression state, if necessary. That is, the wearing time may include the time for which the pressurization state is, that is, the band non-wearing state, although all of the clothing 10 and at least one band of the wearing type correction apparatus 1 for a human body have not been perfectly worn.

When the wearing time is a critical wearing time or more, but pressurization intensity is less than critical pressurization intensity, a patient may be induced to wear the wearing type correction apparatus 1 for a human body normally by further raising only the pressurization intensity because the patient has not given up quite finished wearing the wearing type correction apparatus for a human body.

FIG. 20 illustrates a daily compression rate graph for each hour. In the daily compression rate graph, when the pressure sensor 101 detects arbitrary first compression intensity Pa at arbitrary first timing and detects arbitrary second compression intensity Pb at arbitrary second timing, the compression intensity P between the arbitrary first timing and the arbitrary second timing may be calculated as a value that interpolates the first compression intensity Pa and the second compression intensity Pb. The daily compression rate graph for each hour may be calculated and represented as an average value of at least one measured compression intensity P for each hour.

Furthermore, in the daily compression rate graph for each hour, compression intensity in a normal compression state and compression intensity in a band non-wearing state may be displayed in a pattern form in a predetermined region. A user can visually easily identify whether the curved part of a patient is compressed normally by identifying whether compression intensity is disposed within the pattern region.

FIG. 21 illustrates a daily wearing time graph. The daily wearing time graph may be calculated and illustrated by adding times, that is, the band non-wearing state, and times, that is, the normal compression state, for a day. The daily wearing time graph may include a normal compression state time graph, a band non-wearing state time graph, and a wearing time graph, that is, the sum of a normal compression state time and a band non-wearing state time. Furthermore, in the daily wearing time graph, times equal to or greater than a critical wearing time may be displayed in a pattern form in a predetermined region. Illustratively, a region in which the critical wearing time is 18 hours or more may be displayed in the pattern form. Accordingly, a user can visually easily identify whether a patient observes a sufficient wearing time by identifying whether a wearing time is disposed within the pattern region.

In addition to the aforementioned contents, a weekly wearing time graph and a monthly wearing time graph may be additionally calculated and displayed. A user can visually easily identify a statistical wearing time.

A process of outputting a guide message for improving the pressurization state of a patient to the user terminal based on compliance is described below.

In the method of monitoring corrections using a wearing type correction apparatus for a human body according to an embodiment of the present disclosure, in the display step S150, a guide message for improving the pressurization state of the patient may be additionally output based on compliance that is calculated based on the compression intensity P and the wearing time.

Illustratively, when the average compression intensity P is less than critical compression intensity and when an average wearing time is less than a critical wearing time, it may be determined that a patient has low compliance because the patient has not formed the habits of wearing the wearing type correction apparatus 1 for a human body. In such a case, it is necessary to solicit the patient to wear the wearing type correction apparatus for a human body over a steady wearing time from low compression intensity. Accordingly, in order to improve the pressurization state of the patient in the low compliance, a guide message that is intended to solicit that the time for which the clothing 10 is worn should be increased may be output through the user terminal 300, 400, 500 although the patient does not essentially wear the band.

As another example, when the average compression intensity P is less than critical compression intensity and the average wearing time is critical wearing time or more, it may be determined that a patient has formed the habits of continuously wearing the wearing type correction apparatus 1 for a human body, but has intermediate compliance because the compression intensity P is weak. In such a case, it is necessary to solicit the patient to wear the apparatus with the compression intensity P higher than the existing compression intensity P. Accordingly, in order to improve the pressurization state of the patient in the intermediate compliance, a guide message that is intended to solicit that correction efficiency should be maximized by raising the compression intensity after identifying the wearing of the band may be output through the user terminal 300, 400, 500.

As still another example, when the average compression intensity P is critical compression intensity or more and the average wearing time is a critical wearing time or more, it may be determined that a patient accurately wears the wearing type correction apparatus 1 for a human body and has high compliance. In such a case, it is necessary to solicit the patient to maintain the current wearing habits. Accordingly, in order to maintain the pressurization state of the patient in high compliance, a guide message that is intended to solicit the patient to maintain the wearing habits may be output through the user terminal 300, 400, 500.

There are advantages in that a patient can adjust the apparatus for more effective human body corrections and human body correction efficiency can be maximized because the guide message for improving the pressurization state of a patient is output based on compliance that is calculated based on the compression intensity P and the wearing time as described above.

Meanwhile, in the display step S150, contents displayed on the user terminals 300, 400, and 500 may be different depending on the type of a user. Illustratively, there are advantages in that a patient can visually easily identify whether human body corrections are performed normally through the correction management service app installed in the terminal 300 for a patient, which is possessed by the patient and can be positively motivated with respect to his or her human body corrections through a guide message that is output based on compliance. As another example, there is an advantage in that a psychological safety feel is provided to a guardian of a patient because the guardian can easily identify the wearing state of the patient for the wearing type correction apparatus 1 for a human body although the guardian does not come into contact with the patient, through the correction management service app installed in the terminal 400 for a guardian, which is possessed by the guardian. As still another example, there are advantages in that a doctor can visually easily check statistics that are generated because a patient wears the wearing type correction apparatus 1 for a human body by day, week, and month through the correction management service app installed in the terminal 500 for a doctor and can easily establish a subsequent treatment plan based on the wearing state of the patient.

There is an advantage in that effective treatment for a patient is made possible because information suitable for each user is provided through the user terminal 300, 400, 500 in the display step S150 as described above.

The method of monitoring corrections using a wearing type correction apparatus for a human body according to an embodiment of the present disclosure may further include a releasing step S160. In the releasing step S160, when the pressurization state is analyzed as the excessive compression state, the user terminal 300, 400, 500 or the server 600 may apply a control signal to the wearing type correction apparatus 1 for a human body so that at least one of the curve compressor 100a and the intensity adjuster 100 is controlled. In response to the applied control signal, the amount of winding and unwinding of the wire 105 that connects the intensity adjuster 100 and the curve compressor 100a may be adjusted, and the compression intensity of the curve compressor 100a may be adjusted. That is, when the compression intensity P is the maximum pressure PMAX or more, the pressurization state may be determined to be the excessive compression state, and the wire may be controlled to be loosened so that the pressurization state is adjusted as the normal compression state. Accordingly, compression can be reduced if a patient performs excessive compression on the human body of the patient through the wearing type correction apparatus 1 for a human body. A danger of a pain, a respiratory difficulty, etc. which may be caused by compression on the human body of the patient can be prevented.

As described above, in the wearing type correction apparatus 1 for a human body according to an embodiment of the present disclosure, the pressure sensor 101 is installed in at least one of the curve compressor 100a and the intensity adjuster 100 at a location close to a curved part of a human body, that is, a target to be corrected. Accordingly, the pressure sensor 101 can accurately detect compression intensity. Data including the compression intensity detected by the pressure sensor 101 can be showed to a user through the correction management service app installed in the user terminal 300, 400, 500.

Furthermore, the wearing type correction apparatus 1 for a human body according to an embodiment of the present disclosure can correct the curved part of a patient by adjusting the up and down locations of the curve compressor 100a based on the location of the curved part because the up and down locations of the curve compressor 100a that compresses the curved part can be adjusted.

Furthermore, in the wearing type correction apparatus 1 for a human body according to an embodiment of the present disclosure, a patient can easily adjust the compression intensity of the curve compressor 100a for himself or herself by manipulating the intensity adjuster 100 because the up and down locations of the intensity adjuster 100 that adjusts the compression intensity of the curve compressor 100a can be adjusted.

Furthermore, the wearing type correction apparatus 1 for a human body according to an embodiment of the present disclosure can improve efficiency of the curve compressor 100a that compresses the curved part because the pelvis band 160 holds the pelvis of a patient when the curve compressor 100a compresses the curved part.

Furthermore, the wearing type correction apparatus 1 for a human body according to an embodiment of the present disclosure can improve efficiency of the curve compressor 100a that compresses the curved part because the hump pad 180 is attached to an arbitrary location related to a location at which the curve compressor 100a compresses the curved part and the hump pad 180 compresses the hump part of a human body at an arbitrary location when the curve compressor 100a compresses the curved part.

Furthermore, the wearing type correction apparatus 1 for a human body according to an embodiment of the present disclosure can improve efficiency of the curve compressor 100a that compresses the curved part because the flank band 190 holds the flank of a patient when the curve compressor 100a compresses the curved part.

Furthermore, the wearing type correction apparatus 1 for a human body according to an embodiment of the present disclosure can improve efficiency of the curve compressor 100a that compresses the curved part because the flank pad 200 is attached to an arbitrary location related to a location at which the curve compressor 100a compresses the curved part and the flank pad 200 compresses the arbitrary location when the curve compressor 100a compresses the curved part.

Furthermore, in the wearing type correction apparatus 1 for a human body according to an embodiment of the present disclosure, the wires 183 and 203 of a metal material and the plurality of metal points 215 are displayed in an X-ray photo that is photographed in the state in which the human body correction apparatus 1 has been worn because the wires 183 and 203 are disposed in the hump pad 180 and the flank pad 200 and the metal points 215 are disposed in the point band 210. Accordingly, a doctor can easily treat the curved part of a patient and the location and state of the hump part by monitoring the wires 183 and 203 and the metal points 215 in the X-ray photo.

Meanwhile, an example in which the wearing type correction apparatus 1 for a human body according to an embodiment of the present disclosure has been applied to the clothing 10 that is worn on an upper body of a correction patient has been described, but the present disclosure is not essentially limited thereto and may be applied to a cervical vertebra clothing part that is worn on the cervical vertebra side of a patient, a lumbar vertebrae clothing part that is worn on the lumbar vertebrae side of a patient, an arm clothing part that is worn on an arm or hand of a patient, and a leg clothing part that is worn on a leg or foot of a patient.

Furthermore, in the method of monitoring corrections using a wearing type correction apparatus for a human body according to an embodiment of the present disclosure, whether a patient wears the wearing type correction apparatus for a human body normally can be easily identified by analyzing compression intensity measured by the pressure sensor, and a doctor can establish a corresponding suitable treatment plan.

Furthermore, in the method of monitoring corrections using a wearing type correction apparatus for a human body according to an embodiment of the present disclosure, personalized and optimized monitoring can be performed on a patient because prescription pressure is input and pressurization states are classified and analyzed based on the prescription pressure.

Furthermore, in the method of monitoring corrections using a wearing type correction apparatus for a human body according to an embodiment of the present disclosure, a patient can be induced to correctly wear the apparatus because the guide message capable of improving the pressurization state of the patient is output based on compliance that is calculated based on compression intensity and a wearing time.

Furthermore, in the method of monitoring corrections using a wearing type correction apparatus for a human body according to an embodiment of the present disclosure, inconvenience of a patient can be minimized by adjusting the amount of winding and unwinding of the wire through control of at least one of the curve compressor 100a and the intensity adjuster 100 when it is determined that excessive compression intensity is applied to the human body of the patient.

A person having ordinary knowledge in the art to which the present disclosure pertains will understand that the present disclosure may be implemented in other detailed forms without changing the technical spirit or essential characteristics of the present disclosure. Accordingly, the aforementioned embodiments should be construed as being only illustrative, but should not be construed as being restrictive from all aspects. The scope of the present disclosure is defined by the appended claims rather than by the detailed description, and all changes or modifications derived from the meanings and scope of the claims and equivalent concepts thereof should be interpreted as being included in the scope of the present disclosure.

### INDUSTRIAL APPLICABILITY

The present disclosure provides the wearing type correction apparatus for a human body which can accurately detect compression intensity at a location close to a curved part of a human body, that is, a target to be corrected, and show data including the compression intensity to a user through the correction management service app installed in the user terminal, and a method of monitoring corrections using the same.

1. A wearing type correction apparatus for a human body comprising:
clothing that is worn on an upper body of a patient;
a curve compressor disposed in the clothing and configured to compress a curved part of a human body that is a target to be corrected;
an intensity adjuster disposed in the clothing, connected to the curve compressor through a wire, and configured to adjust compression intensity of the curve compressor by adjusting an amount of winding and unwinding of the wire; and
a pressure sensor installed in at least one of the curve compressor and the intensity adjuster and configured to detect the compression intensity.

2. The wearing type correction apparatus for a human body according to claim 1, further comprising a short-range wireless communication unit installed in at least one of the curve compressor and the intensity adjuster and configured to communicate with a user terminal in which a correction management service app for informing a user of data comprising the compression intensity detected by the pressure sensor is installed.

3. The wearing type correction apparatus for a human body according to claim 1, further comprising a guide rail disposed in the clothing and configured to provide guidance to up and down sliding movements of at least one of the curve compressor and the intensity adjuster.

4. The wearing type correction apparatus for a human body according to claim 3, wherein at least one of the curve compressor and the intensity adjuster comprises:
a body configured to form an appearance shape;
a support plate disposed between the body and the guide rail and combined with the body; and
a rail moving block combined with the support plate and slidably combined with the guide rail.

5. The wearing type correction apparatus for a human body according to claim 4, wherein the intensity adjuster further comprises a dial adjuster rotatably combined with the body and configured to adjust the amount of winding and unwinding of the wire.

6. (canceled)

7. The wearing type correction apparatus for a human body according to claim 1, wherein the rail moving block comprises:
a locking part having a width in the direction orthogonal to the sliding direction of the rail moving block, which is equal to or smaller than the width of the first coupling hole and greater than the width of the second coupling hole, and having one surface come into contact with an outer surface of the support plate;
a support part having a width in the direction orthogonal to the sliding direction of the rail moving block, which is greater than each of the width of the first coupling hole and the width of the second coupling hole, and having one surface that faces the one surface of the locking part come into contact with an inner surface of the support plate; and
a coupling part formed to protrude from other surface of the support part and having a guide groove slidably combined with the guide rail formed therein.

8. The wearing type correction apparatus for a human body according to claim 7, wherein the guide rail comprises:
an attachment plate combined with the clothing; and
a guide protrusion formed on one side of the attachment plate in the direction orthogonal to the sliding direction of the rail moving block and inserted into the guide groove and rotatably disposed therein.

9. (canceled)

10. The wearing type correction apparatus for a human body according to claim 1, wherein:
an opening through which one side of the guide groove is opened is formed in the coupling part, and
an opening width of the opening is formed to be smaller than a diameter of the guide groove.

11-12. (canceled)

13. The wearing type correction apparatus for a human body according to claim 1, further comprising:
a back reinforcement plate disposed on an inner surface of a back part of the clothing; and
a hump pad attached to an arbitrary location related to a location of the back reinforcement plate at which the curve compressor compresses the curved part and configured to compress a hump part of the human body.

14. The wearing type correction apparatus for a human body according to claim 13, further comprising a flank band disposed in the clothing and configured to hold a flank of the patient when the curve compressor compresses the curved part.

15. The wearing type correction apparatus for a human body according to claim 14, further comprising a flank pad attached to an arbitrary location related to a location of the flank band at which the curve compressor compresses the curved part and configured to compress the human body.

16. The wearing type correction apparatus for a human body according to claim 15, wherein the hump pad and the flank pad each comprise:
a sheet-shaped base attached to the back reinforcement plate or the flank band; and
a cushion protrusion formed to protrude on one surface of the base and having cushion power that compresses the human body, and
a wire of a metal material is disposed within an edge of the base.

17. The wearing type correction apparatus for a human body according to claim 1, further comprising a point band attached to an arbitrary location of the clothing and having a plurality of metal points spaced apart from each other up and down disposed therein.

18. (canceled)

19. A method of monitoring a correction state using the wearing type correction apparatus for a human body according to claim 1, the method comprising:
a clothing wearing detection step of detecting, by a pressure sensor installed in at least one of a curve compressor and an intensity adjuster disposed in clothing, first reference pressure in a state in which a patient has worn the clothing;
a band wearing detection step of detecting, by the pressure sensor, second reference pressure in a state in which the patient has worn at least one band disposed in the clothing;
a compression intensity detection step of measuring, by the pressure sensor, compression intensity at predetermined time intervals after the first reference pressure and the second reference pressure are detected; and
a display step of visually displaying the compression intensity through a user terminal.

20. The method according to claim 19, further comprising a pressurization state analysis step of analyzing, by at least one of the user terminal and a server that communicates with the user terminal, a pressurization state of the patient based on the compression intensity,
wherein the pressurization state comprises at least three states based on the first reference pressure and the second reference pressure.

21. The method according to claim 20, wherein the pressurization state comprises:
an excessive compression state in which the compression intensity is equal to or greater than maximum pressure at which the patient does not suffer from a pain;
a normal compression state in which the compression intensity is equal to or greater than average pressure that is an average value of the first reference pressure and the second reference pressure and is less than the maximum pressure;
a band non-wearing state in which the compression intensity is equal to or greater than minimum pressure obtained by subtracting, from the first reference pressure, a deviation between the first reference pressure and the average pressure and is less than the average pressure; and
an apparatus non-wearing state in which the compression intensity is less than the minimum pressure.

22. The method according to claim 20, further comprising a prescription pressure input step of receiving, from the user terminal, prescription pressure separately from the first reference pressure and the second reference pressure,
wherein the pressurization state comprises:
an excessive compression state in which the compression intensity is equal to or greater than maximum pressure at which the patient does not suffer from a pain;
a normal compression state in which the compression intensity is equal to or greater than the prescription pressure and is less than the maximum pressure;
a band non-wearing state in which the compression intensity is equal to or greater than minimum pressure obtained by subtracting, from the first reference pressure, a deviation between the first reference pressure and average pressure that is an average value of the first reference pressure and the second reference pressure and is less than the prescription pressure; and
an apparatus non-wearing state in which the compression intensity is less than the minimum pressure.

23. The method according to claim 21, wherein:
in the display step, a wearing time calculated by using the compression intensity is also displayed, and
the wearing time is calculated by adding times for which the pressurization state is the normal compression state and the band non-wearing state.

24. The method according to claim 23, wherein the display step comprises additionally outputting a guide message for improving the pressurization state of the patient, based on compliance calculated based on the compression intensity and the wearing time.

25. The method according to claim 21, further comprising a releasing step of adjusting the compression intensity of the curve compressor by adjusting an amount of winding and unwinding of a wire that connects the intensity adjuster and the curve compressor through control of at least one of the curve compressor and the intensity adjuster when the pressurization state is analyzed as the excessive compression state.
